(19) United States
(12) Patent Application Publication
(10) Pub. No.: US 2022/0370223 A1
(43) Pub. Date: Nov. 24, 2022

(54) ACTIVE SPLINT FOR TREATING A DISLOCATION OF THE PERINEAL BONE, AND KIT COMPRISING SAME

(71) Applicant: LEVE-TOI ET MARCHE, LA GRANDE MOTTE (FR)

(72) Inventor: Thierry ASTIER, LA GRANDE MOTTE (FR)

(21) Appl. No.: 17/762,068

(22) PCT Filed: Sep. 17, 2020
(86) PCT No.: PCT/FR2020/051608

§ 371 Date: Jun. 6, 2022

(30) Foreign Application Priority Data
Sep. 20, 2019 (FR) FR1910378

(51) Int. Cl. A61F 5/01; A61F 5/058

(52) U.S. Cl. CPC A61F 5/01; A61F 5/0585; A61F 5/058

(57) ABSTRACT

An active splint to treat a dislocation of the perineal bone. The active splint includes a sleeve in which a slide is arranged with the ability to slide between a retracted position and a deployed position. The slide includes a head configured to rest bearing against the head of a perineal bone of a user. An elastic return member is configured to urge the slide towards its retracted position. A blocking member immobilizes the slide and keeps the slide in its deployed position. A detent member is configured to act on the blocking member so as to release the slide from its deployed position.

### TECHNICAL FIELD OF THE INVENTION

The invention relates to the field of medical apparatuses. It relates to treatment and/or rehabilitation apparatuses in particular for a lower limb of a user, and relates more particularly to an active splint allowing in particular correcting the bone position of the perineal bone that has undergone a dislocation and a kit including said active splint.

The splint is so-called “active” to the extent that it is intended to actively act on the user and in particular on his lower limb.

### BACKGROUND OF THE INVENTION

The perineal bone, also called “fibula” in the new nomenclature, forms the lateral portion of the skeleton of the leg. It is articulated at its distal and proximal ends with the tibia and at its distal end with the talus.

In some dysfunction cases, the perineal bone could undergo, by its proximal end, an upward movement at the origin of a joint decoaptation resulting in a dislocation of the perineal bone.

Moreover, the ascent of the upper perineal bone-tibial joint causes the ascent of the lower perineal bone-tibial joint and causes the reduction of the coaptation surface between the talus and the perineal bone. This situation is generally at the origin of a diastasis between the perineal bone and the tibia and/or between the perineal bone and the talus.

Furthermore, a dislocation of the perineal bone might cause the distension of the extensor retinaculum of the foot. In this case, the muscles and their levator tendons of the foot and in particular the tibialis anterior are ineffective in their role of maintaining the tenon-mortise system formed by the talus, the tibia and the perineal bone.

In particular, a dislocation of the perineal bone might generate other pathologies in the affected person, for example, following a phenomenon of compensation of the vertebrae, low back pain, sciatica, crura, lumbago, herniated disc could appear.

In general, the treatment of a dislocation is carried out through the intervention of a medical professional and consists of a manipulation of the leg of the affected person in order to normalise the upper and lower perineal bone-tibial joints.

This treatment is carried out throughout a series of manipulation sessions. The dysfunction on the concerned leg is progressively corrected at each manipulation session but partially reappears between two successive sessions, until it completely disappears.

Consequently, there is a need to treat the dislocation continuously, so as to avoid the patient the pain accompanying this pathology between two consecutive manipulation sessions.

One solution to ensure that the upper and lower peroneotibial joint surfaces are maintained continuously and without any risk of recurrence is to manually normalise the perineal bone, and then apply a compression with plaster or resin.

Nonetheless, this solution is not satisfactory to the extent that it requires the immobilisation of a large portion of the leg, which might complicate walking and generate difficulties in the movements of the person to be treated.

Furthermore, the treatment of the dislocation is incomplete because it does not compensate for the upward and decoaptive component of the biceps femoris which is hypoextensible.

Considering the foregoing, another need for the correction of a dislocation of the perineal bone is the ability to do without a practitioner and to make this correction within the reach of the affected person.

### OBJECT AND SUMMARY OF THE INVENTION

The present invention aims to overcome the aforementioned drawbacks.

To this end, the invention relates to an active splint in particular for the treatment of a dislocation of the perineal bone, including a sheath within which a slider is arranged so as to slidably move between a retracted position and a deployed position, the slider comprising a head intended to bear against the head of a perineal bone of a user, said active splint comprising:
elastic return means configured to urge the slider towards its retracted position,
a member for blocking the slider adapted to hold said slider in its deployed position,
detent means adapted to act on the blocking member so as to release the slider from its deployed position, this release causing driving of the slider into the retracted position by the effect of the elastic return means.

In its travel, the head of the slider, which maintains a constant pressure on the head of the perineal bone, causes the normalisation of the position of the latter.

Hence, thanks to its corrective action, the present invention allows suppressing joint, muscular, bone, dorsal and circulatory nerve pain of the user, and allows correcting any possible related disorders resulting from the dislocation of the perineal bone, while allowing doing without a manipulation by a practitioner.

The present invention also allows compensating for the upward and decoaptive component of the biceps femoris to the extent that when the user is in a seated position, the splint compensates for the decoaptive action of the biceps femoris by applying a radial force against the head of the perineal bone of the user, and during the passage of the user into a standing position, the splint opposes the upward component thanks to the loads of the elastic return means.

In particular embodiments, the invention also responds to the following features, implemented separately or in each of their technically feasible combinations.

In particular embodiments of the invention, the detent means act on the blocking member by the elastic deformation of the sheath and of the slider, or of the slider alone.

In particular embodiments of the invention, the blocking member comprises an elastic tab formed in the slider, comprising at one free end an excrescence adapted to fit in an aperture formed in the sheath.

This blocking member has the advantage of having simple design and manufacture, which positively affects the reliability of the operation of the active splint. Moreover, these features allow participating in the reduction of the dimensions of the active splint.

In particular embodiments of the invention, the sheath includes a recess extending opposite the slider, said recess being configured to receive the excrescence when the slider is in the retracted position and over part of its stroke towards its deployed position, the recess and the aperture being separated by a bridge of material.

This elastic deformation participates in returning a tactile feedback to a user before blocking of the slider in its deployed position.

In particular embodiments of the invention, the detent means are formed by a cantilevered portion of the sheath, extending beyond the aperture, configured to face the elastic tab when the slider is in the deployed position and having elastic properties.

These detent means have the advantage of having simple design and manufacture, which positively affects the reliability of the operation of the active splint. Moreover, these features allow participating in the reduction of the dimensions of the active splint.

In particular embodiments of the invention, the sheath includes an intrados flange intended to bear against a lower limb of a user and an extrados flange opposite to the intrados flange, said flanges being assembled together and being configured to define an inner volume in which the slider is engaged.

In particular embodiments of the invention, the active splint includes guide members enabling the translational guidance of the slider within the sheath, said guide members being formed by ribs extending on the slider and on the sheath and cooperating with each other.

In particular embodiments of the invention, the elastic return means are formed by at least one helical spring enclosed in a cage formed by the guide members, and therefore by ribs.

According to another aspect, the present invention also relates to a kit for the treatment of a dislocation of the perineal bone including an active splint as described before and a passive splint intended to hold the head of the perineal bone in a normal position, said passive splint including:
two support areas connected together by a bridge of material, one of the support areas being intended to be applied against said head of the perineal bone and the other support area being intended to be applied against the bone shaft of the perineal bone,
an elastic link intended to maintain a force applied by said support areas against the lower limb of a user.

According to another aspect, the present invention relates to an active splint or a kit as described before for application thereof in the treatment of a dislocation of the perineal bone.

### BRIEF DESCRIPTION OF THE FIGURES

The invention will be better understood upon reading the following description, given as a non-limiting example, and made with reference to the following figures:

FIG. 1 represents a perspective view of the active splint according to the invention comprising a slider in the retracted position;

FIG. 2 represents a perspective view of a slider of the active splint of FIG. 1;

FIG. 3 represents another perspective view of the slider of FIG. 2;

FIG. 4 represents a perspective view of an extrados flange of a sheath of the active splint of FIG. 1;

FIG. 5 represents a perspective view of an intrados flange of a sheath of the active splint of FIG. 1;

FIG. 6 represents a perspective view of the active splint of FIG. 1 in which the slider is in the deployed position;

FIG. 7 represents a perspective view of the active splint of FIG. 6, in which the intrados flange is represented in transparency;

FIG. 8 represents a perspective view of the active splint of FIG. 1, in which the intrados flange is represented in transparency; and

FIG. 9 represents a perspective view of a passive splint of a kit for treating a dislocation of the perineal bone.

In these figures, reference numerals that are identical from one figure to another refer to identical or similar elements. Moreover, for clarity, the drawings are not plotted to scale, unless stated otherwise.

### DESCRIPTION OF THE EMBODIMENTS

The present invention relates to an active splint 10 intended to move the head of the perineal bone of a user to bring it back to its normal position, in particular following a dislocation.

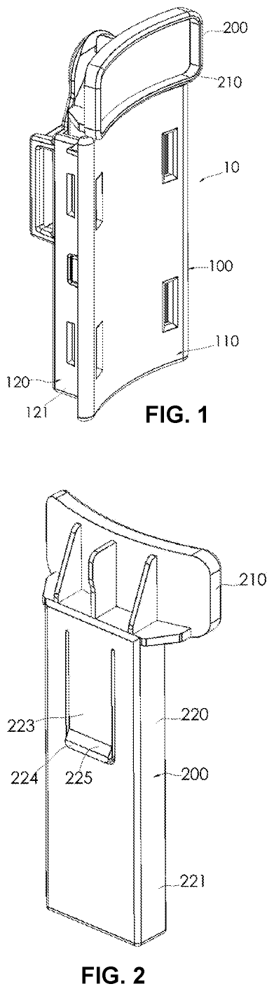

As shown in FIG. 1, the active splint 10 includes a sheath 100 with which a slider 200 slidably cooperates along a translation axis, between an extended position and a retracted position.

The sheath 100 and the slider 200 extend along the same longitudinal axis parallel to or coincident with the axis of translation of the slider 200.

The splint 10 is intended to be pressed laterally, directly or indirectly, against the teguments of the leg of the user by continuously maintaining a pressure against the latter and more particularly against the perineal bone of the user. The perineal bone being linked to the tibia, this force is also transmitted to the tibia through the perineal bone and allows correcting the dysfunction related to the dislocation as described in more detail hereinafter.

To this end, the active splint 10 may include a link (not represented in the figures), preferably elastic, intended to surround the leg of the user in order to hold the splint against the latter at a constant pressure with a predefined intensity. For example, the link includes two free ends each of which includes fastening members intended to cooperate with each other. These fastening members may be formed by textile hooks and loops.

In the preferred embodiment of the invention represented in FIGS. 1 and 4 to 8, the sheath 100 is formed by two flanges or half-shells, respectively called “intrados flange” 110 and “extrados flange” 120, the intrados flange 110 being intended to be fastened against the lower limb of a user and the extrados flange 120 being intended to be arranged opposite the outside environment.

The intrados 110 and extrados 120 flanges are configured to form an inner volume receiving the slider 200 and respectively comprise an inner face and an outer face, said outer faces being directed towards the outside environment and the inner faces being directed towards the inner volume.

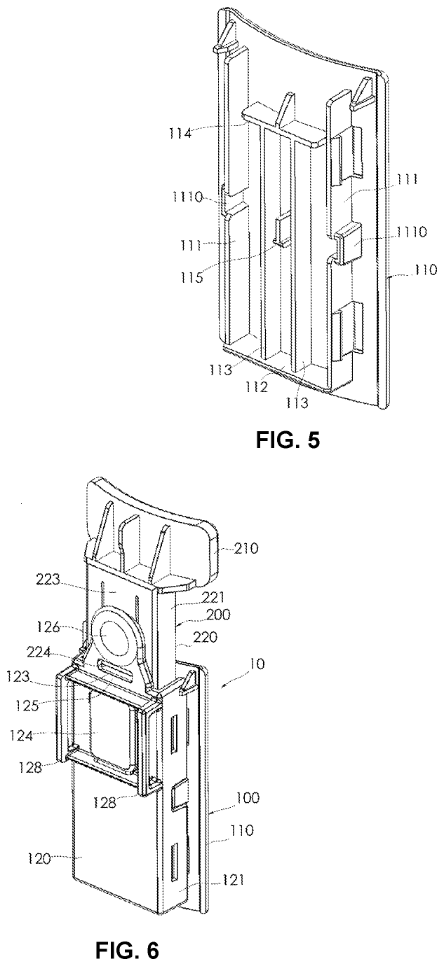

More specifically, as visible in particular in FIGS. 4 and 5, in order to define the inner volume, the intrados 110 and extrados 120 flanges respectively include two parallel lateral walls forming sidewalls 111 and 121, extending from their inner face. The sidewalls 111 of the intrados flange 110 are configured to cooperate with the sidewalls 121 of the extrados flange 120 by elastic nesting.

In the embodiment represented in FIGS. 1 and 4 to 8, the sidewalls 121 of the extrados flange 120 may comprise notches 1210 into which are intended to be fitted protuberances 1110 extending from the inner face of the intrados flange 110. Advantageously, the interlocking of the protuberances 1110 with the notches 1120 forms a fool proof device facilitating the assembly of the intrados 110 and extrados 120 flanges with each other.

As shown in FIGS. 4 and 5, the sidewalls 111 of the intrados flange 110 are connected to each other by a transverse bottom wall 112. Similarly, the sidewalls 121 of the extrados flange 120 are connected to each other by a bottom wall 122.

In the embodiment of the invention represented in FIGS. 1 and 6 to 8, the sidewalls 111 of the intrados flange 110 are arranged opposite the inner volume and the sidewalls 121 of the extrados flange 120 are arranged opposite the outer volume when the sheath is assembled.

The outer face of the intrados flange 110 is intended to be applied against the user and advantageously has a cross-section with a concave shape for this purpose.

Advantageously, the outer face of the extrados flange 120 comprises two loops 128 through the link intended to surround the leg of the user is engaged.

In the embodiment represented in FIGS. 1 and 4 to 8, the slider 200 is housed between the sidewalls 111 and 121 of the intrados 110 and extrados 120 flanges.

It has the shape of a thin part and includes a head 210 connected to a body 220.

As shown in FIGS. 1 and 6 to 8, the body 220 of the slider 200 is engaged in the inner volume of the sheath 100 and its head 210 extends outside of said sheath 100.

In the present text, the term “inner end” of the slider 200 designates the free end of the body 220 of the slider 200.

The head 210 of the slider 200 is intended to be applied directly or indirectly against the integuments of a patient, and more particularly against the head of the perineal bone of the user.

Advantageously, the head 210 of the slider 200 may have a cross-section with a concave shape, for example according to a radius substantially identical to the cross-section of the outer face of the intrados flange 110, so as to conform to the shape of the leg of the patient. The head 210 of the slider 200 and more particularly its surface intended to face the leg of the user may be covered with a dense foam layer, for example fastened by gluing.

The slider 200 includes a face called the “intrados face” intended to be directed towards the leg of the user, opposite to a face called the “extrados face” intended to be directed towards the outside environment.

Advantageously, the active splint 10 includes guide members allowing guiding the slider 200 in translation within the sheath 100.

Advantageously, these guide members may be formed by ribs extending on the slider 200 and on the sheath 100, said ribs of the slider 200 being intended to cooperate with those of the sheath 100.

FIGS. 2, 3, 6 to 8 represent an embodiment wherein these guide members are formed in particular by two parallel lateral walls 221 facing each other and extending along the body 220 of the slider 200, on either side thereof.

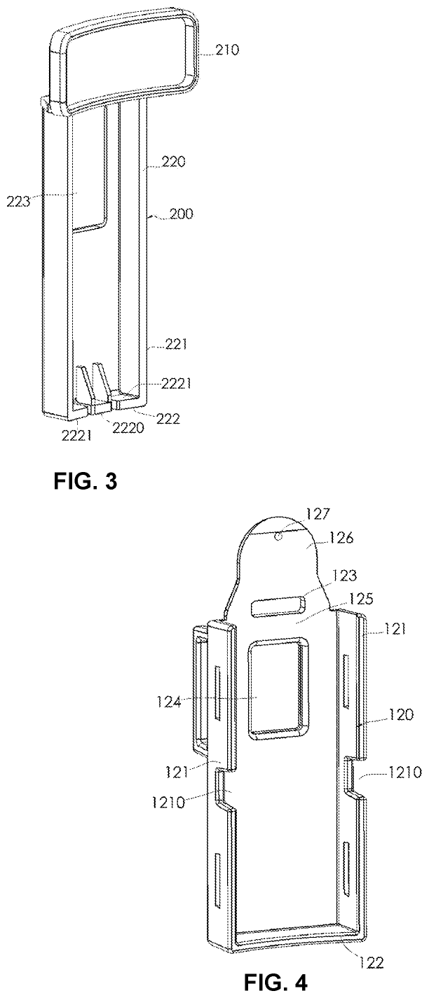

These two lateral walls 221 preferably extend from the head 210 of the slider 200 up to its inner end at which they are connected to each other by an end transverse wall 222, as visible in FIGS. 3 and 8.

In the present text, the term “transverse” relates to a direction perpendicular to a direction defined by the longitudinal axis of the sheath 100 and of the slider 200.

Preferably, the guide members are also formed by the sidewalls 111 of the intrados flange 110, each of the lateral walls 221 of the slider 200 being intended to slip against one of said sidewalls 111 of the intrados flange 110 during sliding of the slider.

Advantageously, the sidewalls 111 of the intrados flange 110 and the lateral walls 221 of the slider 200 are sized so as to prevent any angular displacement of said slider 200.

As represented in FIGS. 6, 7 and 8 in a preferred embodiment, the guide members may also comprise two parallel longitudinal middle walls 113 extending longitudinally on the inner face of the intrados flange 110, said longitudinal middle walls 113 being arranged at a distance from each other so as to form a middle groove. At one of their ends, the longitudinal middle walls 113 are extended by the bottom wall 112 of the intrados flange 110 and are extended at the other end by a transverse middle wall 114 of the intrados flange 110.

In order to guide sliding of the slider 200 within the sheath 100, the end transverse wall 222 includes a recess in line with each of the longitudinal middle walls 113. Thus, and as shown in particular in FIGS. 3 and 8, the end transverse wall 222 is split into three portions: a middle portion 2220 interposed between two lateral portions 2221.

The active splint 10 includes elastic return means configured to urge the slider 200 towards the inside of the sheath 100, i.e. to retract it.

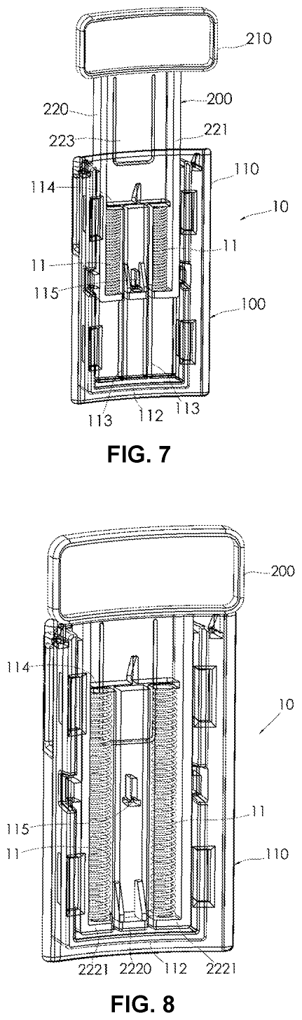

In the example represented in FIGS. 7 and 8, the elastic return means are formed by two helical springs 11 working in compression.

In other embodiments, the elastic return means may be formed by at least one helical spring.

Each helical spring 11 bears by one of its ends, against one of the lateral portions 2221 of the end transverse wall 222, and by its other end, against the bottom wall 112 of the intrados flange 110.

As shown in FIGS. 7 and 8, each of the helical springs 11 is advantageously enclosed in a cage formed by the guide members, and therefore by ribs. More particularly, each cage is formed by the combination of a sidewall 111 of the intrados flange 110, of a lateral portion 2221 of the end transverse wall 222, of a longitudinal middle wall 113 and of a portion of the transverse middle wall 114 of the intrados flange 110. Hence, the helical springs 11 are arranged on either side of the middle groove.

Hence, to drive the slider 200 in the deployed position, the user has to overcome the return forces exerted by the helical springs 11.

Advantageously, the intrados flange 110 includes a middle stop 115 intended to rest against the middle portion 2220 of the end transverse wall 222 when the slider 200 is in its deployed position, as shown in FIG. 7.

Hence, the function of this middle stop 115 is to define the maximum stroke of the slider 200 in extension.

The bottom wall 112 of the intrados flange 110 advantageously forms a stop defining the retracted position of the slider 200, in which the end transverse wall 222 bears against the bottom wall 112 of the intrados flange 110.

The active splint 10 includes a member for blocking the slider 200 adapted to hold said slider 200 in its deployed position, as represented in FIGS. 6 and 7.

This blocking member is configured to be automatically actuated during the movement of the slider 200 towards its deployed position, and more specifically, when it reaches its deployed position.

To this end, as shown in particular in FIGS. 2, 3 and 6, the slider 200 includes an elastic tab 223 comprising an excrescence 224 at its free end, said elastic tab 223 being configured so that its excrescence 224 cooperates with an aperture 123 with which the extrados flange 120 is provided on its inner face.

In the embodiment represented in FIGS. 4 and 6, the aperture 123 is open-through.

In other words, the excrescence 224 is adapted to fit in said aperture 123 by deformation of the elastic tab 223 when the slider 200 occupies its deployed position.

Advantageously, the extrados flange 120 includes a recess 124 on its inner face, over a thickness corresponding at least to the thickness of the excrescence 224 with respect to the extrados face of the slider; said recess 124 being arranged so that, when the slider 200 is in the retracted position and over a portion of its stroke towards its deployed position, the excrescence 224 fits within the recess 124 and the elastic tab 223 is in a rest position, i.e. it does not undergo any deformation.

In other words, the recess 124 is configured so as to allow housing the excrescence 224 of the elastic tab 223 during a portion of its movement from the retracted position of the slider 200 towards its deployed position.

Preferably, the recess 124 and the aperture 123 are separated by a bridge of material 125 as shown in FIGS. 4 and 6.

Advantageously, the excrescence 224 has a cross-section included in a plane parallel to the longitudinal axis of the slider 200, a portion 225 of which decreases up to the surface of the elastic tab 223, as illustrated in FIG. 2.

This decreasing portion 225 forms a bearing surface against the bridge of material 125 so that when the slider 200 is driven towards its deployed position, the elastic tab 223 is progressively deformed.

In the remainder of the stroke of the slider 200 towards its deployed position, the excrescence 224 crosses the bridge of material 125 by slipping against it, the elastic tab 223 then undergoing a maximum deformation state, until said excrescence 224 lies opposite the aperture 123 into which it is forced to fit by the force generated by the elastic return of said elastic tab 223, which then recovers its rest position.

This elastic deformation participates in transmitting tactile feedback to a user before and during the blocking of the slider 200 in its deployed position.

In order to release the slider 200 from its deployed position, the active splint 10 includes detent means adapted to act on the blocking member. When it is no longer immobilised in the deployed position, the slider 200 is driven towards its retracted position by the return force generated by the helical springs 11.

Advantageously, the user actuates the detent means to generate a force on the head of the perineal bone against which the head 210 of the slider 200 is intended to rest, in order to move said head of the perineal bone towards its normal position.

Advantageously, in the preferred embodiment of the invention, the detent means act on the blocking member by the elastic deformation of the sheath 100 and of the slider 200, more particularly of the elastic tab 223.

As represented in FIGS. 1, 4 and 6, the extrados flange 120 includes a cantilevered portion 126 extending beyond the inner volume. This cantilevered portion 126 is configured to face the elastic tab 223 when the slider 200 is in the deployed position. The cantilevered portion 126 advantageously has elastic properties, so that by applying a sufficient pushing force on the latter, the user can cause its deformation in order to apply a pushing force on the elastic tab 223 to cause the deformation thereof and dislodge the excrescence 224 from the aperture 123.

Thus, by applying a pushing force on the cantilevered portion 126 of the outer flange, the user can release the slider 200 from its deployed position and cause the movement thereof towards its retracted position by the return forces generated by helical springs 11.

In FIGS. 4 and 6, the aperture 123 is formed on the cantilevered portion 126. Nonetheless, in another embodiment of the invention, the aperture 123 could be formed at another location of the extrados flange 120 without affecting the aforementioned operation of the invention.

Advantageously, the cantilevered portion 126 may comprise a hemispherical stud 127 as visible in FIG. 4, or any other type of projection, extending towards the elastic tab 223.

This stud 127 allows concentrating the forces applied by the cantilevered portion 126 on the elastic tab 223 and therefore reducing the frictions between the cantilevered portion 126 and said elastic tab 223, and thus allows facilitating the deformation thereof.

In another embodiment (not represented in the figures), the cantilevered portion 126 has no elastic property and the detent means are formed by a pushbutton crossing said cantilevered portion 126, said pushbutton being configured to be able to apply a pushing force on the elastic tab 223 so as to dislodge the excrescence 224 from the aperture 123.

In this embodiment, the detent means act on the blocking member by elastic deformation of the slider 200 alone.

The present invention is integrated according to another aspect, in a kit for treating a dislocation of the perineal bone.

The kit includes, in addition to the active splint 10, a passive splint 20, intended to be used following the use of the active splint 10, complementarily with the latter.

More particularly, the passive splint 20 described hereinafter is provided to hold the head of the perineal bone in its normal position by the active splint 10 in order to modify the memory of the ligament structures.

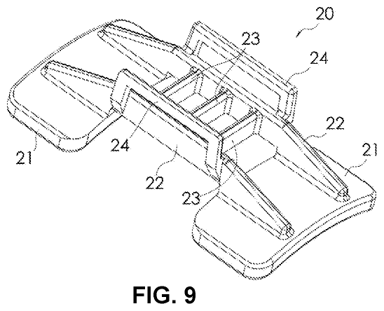

As shown in FIG. 9 in one embodiment, the passive splint 20 includes two support areas 21 connected together by two parallel branches 22.

These parallel branches are joined to each other by transverse ribs 23. These transverse ribs 23 allow increasing the mechanical strength of the passive splint 20 while participating in the reduction of its mass.

The passive splint 20 further includes loops 24 intended to receive an elastic link (not represented in the figures) in order to hold said passive splint 20, and more particularly its support areas 21, against the leg of the user.

All of the surfaces of the active splint 10 and of the passive splint 20 intended for direct or indirect contact with the integuments of a user may advantageously be covered with a hypoallergenic textile.

Moreover, the sheath 100 and the slider 200 of the active splint 10 as well as the passive splint 20 may be made of thermoformable material.

More generally, it should be noted that the implementations and embodiments of the invention considered hereinabove have been described as non-limiting examples and that other variants could consequently be considered

1-9. (canceled)

10. An active splint comprising:
a sheath within which a slider is arranged so as to slidably move between a retracted position and a deployed position, the slider comprising a head configured to bear against a head of a perineal bone of a user;
an elastic return member configured to urge the slider towards the retracted position;
a blocking member to block the slider and configured to hold the slider in the deployed position; and
a detent member configured to act on the blocking member so as to release the slider from the deployed position.

11. The active splint of claim 10, wherein the detent member acts on the blocking member by an elastic deformation of the sheath and of the slider.

12. The active splint of claim 10, wherein the blocking member comprises an elastic tab formed in the slider, the elastic tab comprising at one free end an excrescence configured to fit in an aperture formed in the sheath.

13. The active splint of claim 12, wherein the sheath comprises a recess extending opposite the slider, the recess being configured to receive the excrescence when the slider is in the retracted position and over part of a slider's stroke towards the deployed position, the recess and the aperture being separated by a bridge of material.

14. The active splint of claim 12, wherein the detent member is formed by a cantilevered portion of the sheath, extending beyond the aperture, configured to face the elastic tab when the slider is in the deployed position and the cantilevered portion having elastic properties.

15. The active splint of claim 10, wherein the sheath comprises an intrados flange configured to bear against a lower limb of the user, and an extrados flange opposite to the intrados flange, the intrados and extrados flanges being assembled together and being configured to define an inner volume in which the slider is engaged.

16. The active splint of claim 10, further comprising guide members to enable a translational guidance of the slider within the sheath, the guide members being formed by ribs extending on the slider and on the sheath, the guide members cooperating with each other.

17. The active splint of claim 16, wherein the elastic return member is formed by at least one helical spring enclosed in a cage formed by the guide members.

18. A kit comprising the active splint of claim 10 and a passive splint configured to hold the head of the perineal bone in a normal position, the passive splint comprising:
two support areas connected together by a bridge of material, one of the two support areas being configured to be applied against the head of the perineal bone and other support area being configured to be applied against a bone shaft of the perineal bone; and
an elastic link configured to maintain a force applied by the two support areas against a lower limb of the user.
